(19) United States
(12) Patent Application Publication
(10) Pub. No.: US 2023/0256208 A1
(43) Pub. Date: Aug. 17, 2023

(54) Vascular Access System with Flow Restriction Device

(71) Applicant: Becton, Dickinson and Company, Franklin Lakes, NJ (US)

(72) Inventor: Yiping Ma, Layton, UT (US)

(21) Appl. No.: 18/110,086

(22) Filed: Feb. 15, 2023

Related U.S. Application Data
(60) Provisional application No. 63/310,894, filed on Feb. 16, 2022.

(51) Int. Cl. A61M 25/06; A61M 25/00

(52) U.S. Cl. CPC A61M 25/0606; A61M 25/0097; A61M 2202/0413; A61M 2205/3334

(57) ABSTRACT

A vascular access system includes a catheter adapter having a body and a catheter configured to be inserted into a patient's vasculature, with the body having a distal end and a proximal end positioned opposite the distal end, and a blood collection device in fluid communication with the catheter. The blood collection device including a flow restriction device configured to reduce a blood collection flow rate to be equal to or less than a blood supply flow rate of a patient's blood vessel.

### CROSS-REFERENCE TO RELATED APPLICATION

The present application claims priority to U.S. Provisional Application No. 63/310,894, entitled “Vascular Access System with Flow Restriction Device”, filed Feb. 16, 2022, the entire disclosure of which is hereby incorporated by reference in its entirety.

### BACKGROUND OF THE INVENTION

### Field of the Invention

The present disclosure relates to a vascular access system with a flow restriction device.

### Description of Related Art

Catheters are frequently utilized to administer fluids into and out of the body. Patients in a variety of settings, including in hospitals and in home care, receive fluids, pharmaceuticals, and blood products via a vascular access device inserted into a patient's vascular system. Catheters of various types and sizes have been used extensively in a variety of procedures including, but not limited to, treating an infection, providing anesthesia or analgesia, providing nutritional support, treating cancerous growths, maintaining blood pressure and heart rhythm, and many other clinical uses. A common vascular access device is a plastic catheter that is inserted into a patient's vein. The catheter length may vary from a few centimeters for peripheral access to many centimeters for central access. The catheter is commonly incorporated into a catheter adapter to aid in the ease of use, accessibility and utility of the catheter. A catheter adapter may be adapted to house one end of the catheter such that one end of the catheter is supported by the catheter adapter and the body and tip of the catheter extends beyond a first end of the catheter adapter. A catheter adapter generally further includes a second end adapted to receive additional infusion components for use with the catheter. For example, the second end of a catheter adapter may include a set of threads for attaching an intravenous line or for coupling a syringe to the catheter adapter thereby providing access to the patient's vasculature via the attached catheter.

The catheter may be inserted transcutaneously. When inserted transcutaneously, the insertion of the catheter is commonly aided by an introducer needle. The introducer needle is commonly housed inside the lumen of the catheter such that the gauge of the needle approximates the inner diameter of the catheter. The needle is positioned within the catheter such that the needle tip extends beyond the tip of the catheter whereby the needle is used to penetrate the patient's vein and provide an opening for insertion of the catheter.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal, fluid infusion, or probe access.

Blood withdrawal or infusion using the catheter may be difficult for several reasons, particularly when a dwelling time of the catheter within the patient is more than one day. For example, when the catheter is left inserted in the patient for a prolonged period of time, the catheter may be more susceptible to narrowing, collapse, kinking, blockage by debris (e.g., fibrin, platelet clots, or thrombus), and adhering of a tip of the catheter to the vasculature. Due to this, catheters may often be used for acquiring a blood sample at a time of catheter placement but are much less frequently used for acquiring a blood sample during the catheter dwell period. Therefore, when a blood sample is desired, an additional needle stick is used to provide vein access for blood collection, which may be painful for the patient and result in higher material costs.

### SUMMARY OF THE INVENTION

In one aspect or embodiment, a vascular access system includes a catheter adapter that includes a body and a catheter configured to be inserted into a patient's vasculature, with the body having a distal end and a proximal end positioned opposite the distal end, and a blood collection device in fluid communication with the catheter, with the blood collection device including a flow restriction device configured to reduce a blood collection flow rate to be equal to or less than a blood supply flow rate of a patient's blood vessel.

The flow restriction device may be a flexible extension set including tubing. An inner diameter and length of the tubing may be configured to reduce the blood collection flow rate. The flow restriction device may be configured to reduce a max shear stress compared to a max shear stress of the catheter.

The flow restriction device may have a geometric factor, G_{f}, configured to deliver a predetermined blood collection flow rate reduction, where Gt=L/D⁴, where L is a length of a flow path of the flow restriction device, and where D is an inner diameter of the flow restriction device. The geometric factor, G_{f}, may be at least 3.87 E6 to 8.38 E6. The geometric factor, G_{f}, may be at least 3.87 E6. The geometric factor, G_{f}, may be at least 4.35 E6. The geometric factor, G_{f}, may be at least 5.80 E6. The geometric factor, G_{f}, may be at least 8.38 E6.

The blood collection device may include a luer lock access device configured to receive an evacuated blood collection container. The blood collection device may include a luer connector configured to be connected to a syringe barrel.

The vascular access system may include an instrument advancement device coupled to the catheter adapter, where the instrument advancement device includes an instrument, with the instrument advancement device configured to advance the instrument from a retracted position to an advanced position beyond a distal end of the catheter. The vascular access system may include an advancement member configured to be grasped by a healthcare technician, where movement of the advancement member moves the instrument between the retracted position and the advanced position.

In a further aspect or embodiment, a vascular access system includes a catheter adapter including a body and a catheter configured to be inserted into a patient's vasculature, with the body having a distal end and a proximal end positioned opposite the distal end, a first blood collection device configured to be in fluid communication with the catheter, with the first blood collection device including a first flow restriction device configured to reduce a blood collection flow rate to a first predetermined blood collection flow rate, and a second blood collection device configured to be in fluid communication with the catheter, with the second blood collection device including a second flow restriction device configured to reduce a blood collection flow rate to a second predetermined blood collection flow rate. The first predetermined blood collection flow rate is larger than the second predetermined blood collection flow rate.

In a further aspect or embodiment, a method of selecting a blood collection device for use with a catheter adapter including a body and a catheter configured to be inserted into a patient's vasculature, includes: providing a first blood collection device configured to be in fluid communication with the catheter, with the first blood collection device including a first flow restriction device configured to reduce a blood collection flow rate to a first predetermined blood collection flow rate; providing a second blood collection device configured to be in fluid communication with the catheter, with the second blood collection device including a second flow restriction device configured to reduce a blood collection flow rate to a second predetermined blood collection flow rate, where the first predetermined blood collection flow rate is larger than the second predetermined blood collection flow rate; and selecting the first blood collection device or the second blood collection device based on an estimated blood supply flow rate of a patient's blood vessel.

In a further aspect or embodiment, a blood collection device includes a flow restriction device configured to reduce a blood collection flow rate to be equal to or less than a blood supply flow rate of a patient's blood vessel.

The flow restriction device may be a flexible extension set including tubing. An inner diameter and length of the tubing may be configured to reduce the blood collection flow rate. The flow restriction device may be configured to reduce a max shear stress compared to a max shear stress of the catheter. The blood collection device may include a luer lock access device configured to receive an evacuated blood collection container. The blood collection device may include a luer connector configured to be connected to a syringe barrel.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a vascular access system according to one aspect or embodiment of the present application, showing a retracted position of an instrument;

FIG. 2 is a perspective view of a vascular access system according to a further aspect or embodiment of the present application, showing an extended positon of an instrument;

FIG. 3 is a perspective view of a flow restriction device according to one aspect or embodiment of the present application;

FIG. 4 is a perspective view of a flow restriction device according to one aspect or embodiment of the present application;

FIG. 5 is a graph comparing a sum of blood collection rate with a gauge-specific flow restriction device to achieve a 2× reduction in a blood collection flow rate to a sum of blood collection rate without a flow restriction device for 18G, 20G, 22G, and 24G blood collection needles or catheters;

FIG. 6 is a graph comparing a sum of max shear stress (as a ratio to a max shear stress of a known 21G blood collection needle) with a gauge-specific flow restriction device to achieve a 2× reduction in a blood collection flow rate to a sum of blood collection rate without a flow restriction device for 18G, 20G, 22G, and 24G blood collection needles or catheters;

FIG. 7 is a graph comparing a sum of blood collection rate with a gauge-specific flow restriction device to achieve a 3× reduction in a blood collection flow rate to a sum of blood collection rate without a flow restriction device for 18G, 20G, 22G, and 24G blood collection needles or catheters;

FIG. 8 is a graph comparing a sum of max shear stress (as a ratio to a max shear stress of a known 21G blood collection needle) with a gauge-specific flow restriction device to achieve a 3× reduction in a blood collection flow rate to a sum of blood collection rate without a flow restriction device for 18G, 20G, 22G, and 24G blood collection needles or catheters;

FIG. 9 is a graph comparing a sum of blood collection rate (as a ratio to a blood collection rate of a known 21G blood collection needle) with a gauge-specific flow restriction device to achieve a 3× reduction in a blood collection flow rate relative to a known 21G blood collection needle to a sum of blood collection rate without a flow restriction device for 18G, 20G, 22G, and 24G blood collection needles or catheters; and

FIG. 10 is a graph comparing a sum of max shear stress (as a ratio to a max shear stress of a known 21G blood collection needle) with a gauge-specific flow restriction device to achieve a 3× reduction in a blood collection flow rate relative to a known 21G blood collection needle to a sum of blood collection rate without a flow restriction device for 18G, 20G, 22G, and 24G blood collection needles or catheters.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

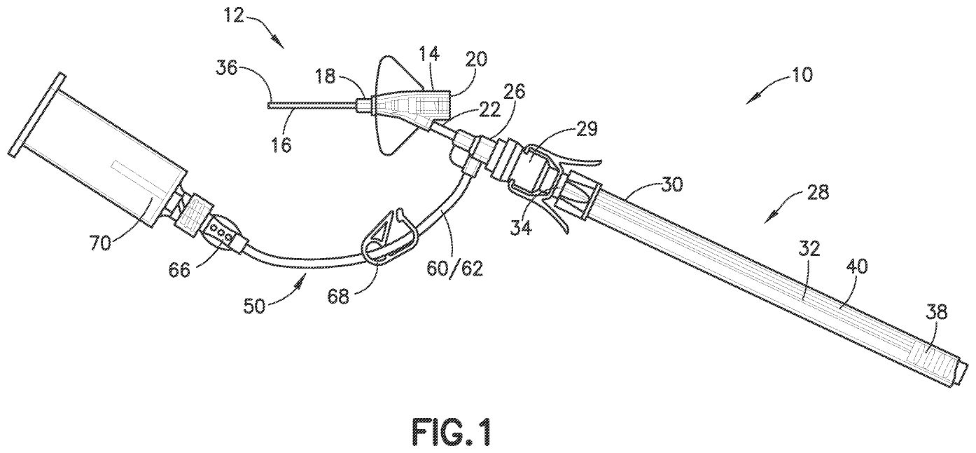

Referring to FIGS. 1-3, in one aspect or embodiment, a vascular access system 10 includes a catheter assembly 12, which may include a catheter adapter 14 and a catheter 16. The catheter 16 may be a peripheral intravenous catheter, a peripherally-inserted central catheter, or a midline catheter. In some aspects or embodiments, the catheter adapter 14 includes a distal end 18, a proximal end 20, and a lumen extending through the distal end 18 and the proximal end 20. In some aspects or embodiments, the catheter 16 extends distally from the distal end 18 of the catheter adapter 14. The catheter adapter 14 may be integrated with an extension tube 22, which may extend from a side port of the catheter adapter 14. In some aspects or embodiments, an adapter 26, such as a Y-adapter or a T-adapter, for example, may be coupled to a proximal end of the extension tube 22. An instrument advancement device 28 may be coupled to the catheter assembly 12 in various ways. In one aspect or embodiment, the instrument advancement device 28 is coupled to a port of the adapter 26. In one aspect or embodiment, the instrument advancement device 28 is coupled to a needleless connector 29 disposed between the port of the adapter 26 and the instrument advancement device 28. The instrument advancement device 28 may also be coupled to the proximal end 20 of the catheter adapter 14.

The instrument advancement device 28 may include a housing 30 configured to couple to the catheter assembly 12. The instrument advancement device 28 includes an instrument 32. In some aspects or embodiments, the instrument advancement device 28 may include any suitable delivery device. Some examples of instrument advancement devices that may be used with the instrument 32 are described further in in U.S. patent application Ser. No. 16/037,246, filed Jul. 17, 2018, entitled “EXTENSION HOUSING A PROBE OR INTRAVENOUS CATHETER,” U.S. patent application Ser. No. 16/388,650, filed Apr. 18, 2019, entitled “INSTRUMENT DELIVERY DEVICE HAVING A ROTARY ELEMENT,” U.S. patent application Ser. No. 16/037,319, filed Jul. 17, 2018, entitled “MULTI-DIAMETER CATHETER AND RELATED DEVICES AND METHODS,” U.S. patent application Ser. No. 16/502,541, filed Jul. 3, 2019, entitled “DELIVERY DEVICE FOR A VASCULAR ACCESS INSTRUMENT,” U.S. patent application Ser. No. 16/691,217, filed Nov. 21, 2019, entitled “SYRINGE-BASED DELIVERY DEVICE FOR A VASCULAR ACCESS INSTRUMENT,” U.S. Patent Application No. 62/794,437, filed Jan. 18, 2019, entitled “CATHETER DELIVERY DEVICE AND RELATED SYSTEMS AND METHODS,” and U.S. Patent Application No. 62/830,286, filed Apr. 5, 2019, entitled “VASCULAR ACCESS INSTRUMENT HAVING A FLUID PERMEABLE STRUCTURE AND RELATED DEVICES AND METHODS,” which are each incorporated by reference in their entirety.

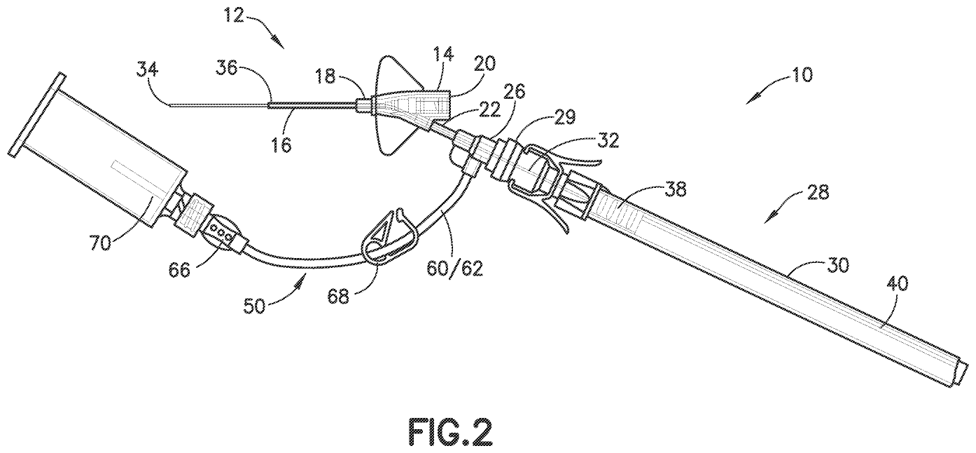

In some aspects or embodiments, the instrument advancement device 28 may be configured to introduce the instrument 32 into the catheter assembly 12. In response to the instrument 32 being introduced into the catheter assembly 12, the instrument 32 may access a fluid path of the catheter assembly 12 and/or the instrument 32 may extend through the catheter assembly 12 to access the vasculature of a patient. The instrument advancement device 28 may be configured to advance the instrument 32 between a retracted position, illustrated, for example, in FIG. 1, to an advanced position, illustrated, for example, in FIG. 2. A distal tip 34 of the instrument 32 may be disposed distal to a distal end 36 of the catheter 16 in response to the instrument 32 being in the advanced position. In response to the instrument 32 being in the retracted position, the distal tip 34 of the instrument 32 may be disposed within the housing 30. A proximal end of the instrument 32 may be coupled to an advancement tab 38, which may be gripped and moved along a slot 40 by a user to move the instrument 32 between the retracted position and the advanced position. The advancement tab 38 may extend through the slot 40, and a portion of the advancement tab 38 coupled to the proximal end of the instrument 32 may be within the housing 30.

Referring again to FIGS. 1-3, the vascular access system 10 includes a blood collection device 50 in fluid communication with the catheter 16. As discussed in more detail below, the blood collection device 50 includes a flow restriction device 60 configured to reduce a blood collection flow rate to be equal to or less than a blood supply flow rate of a patient's blood vessel. The blood collection device 50 may be coupled to a port of the adapter 26, although other suitable configurations may be utilized. During blood draw using the vascular access system 10, such as through the use of an evacuated blood collection tube (not shown), the blood supply may be limited if the catheter 16 was placed near the extremities of arms of the patient. Similarly, the blood flow could also be limited in pediatric patients or patients who are dehydrated. With patients having a weak vessel, the blood vessel may collapse when the blood flow is limited during a blood draw. All of these circumstances may lead to blood draw difficulty or failure. The flow restriction device 60 of the present application is configured to ensure blood draw success in patients where blood flow in their vasculature is limited.

Referring to FIG. 3, in one aspect or embodiment, the flow restriction device 60 of the blood collection device is a flexible extension set including tubing 62. The blood collection device 50 includes a male luer connector 64 at one end of the tubing 62 and a female luer connector 66 at the other end of the tubing 62. The blood collection device 60 may include a tube clamp 68. The female luer connector 66 may be connected to a syringe barrel (not shown) or other suitable blood collection container. In certain aspects or embodiments, the flow restriction device 60 is a flexible extension set, a compact connector with a serpentine flow path, and/or a rigid connector with an elongated fluid path.

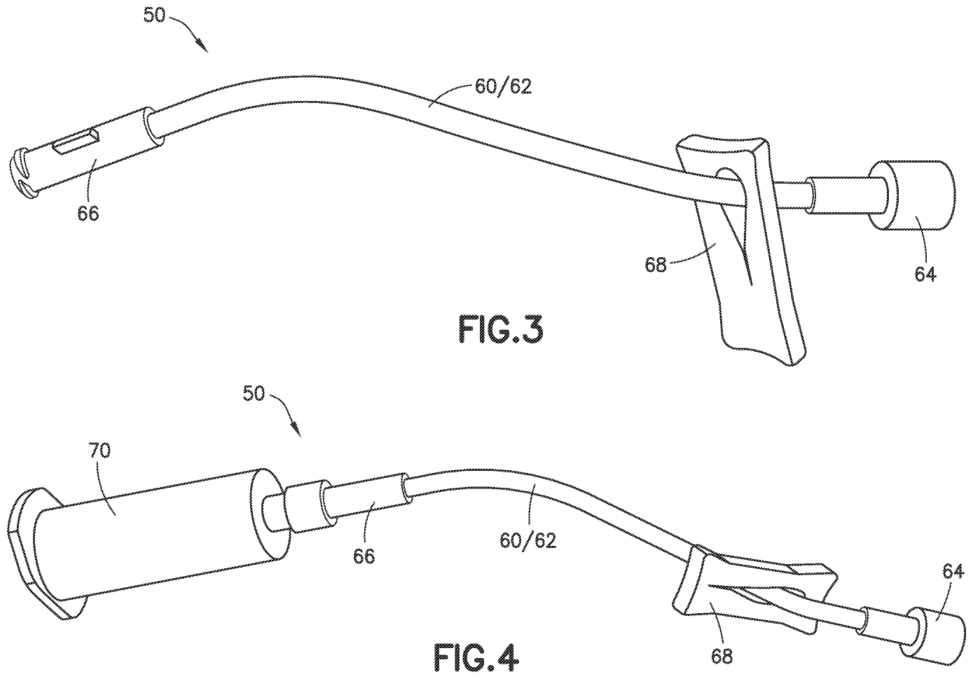

Referring to FIG. 4, in one aspect or embodiment, the blood collection device 50 includes a luer lock access device 70 configured to receive an evacuated blood collection container.

In one aspect or embodiment, an inner diameter and length of the tubing of the flow restriction device 60 is configured to reduce the blood collection flow rate. In one aspect or embodiment, the flow restriction device 60 is also configured to reduce a max shear stress compared to a max shear stress of the catheter 16.

In one aspect or embodiment, the flow restriction device 50 has a geometric factor, G_{f}, configured to deliver a predetermined blood collection flow rate reduction. The geometric factor, G_{f}, is calculated using the equation: Gt=L/D⁴ for a simple fluid path where L is a length of a flow path of the flow restriction device 60 and where D is an inner diameter of the flow restriction device 60. The minimum geometric factor, G_{f}, to achieve at least a 2× reduction in blood collection flow based on a gauge of the catheter or needle is shown in Table 1 below:

TABLE 1

| Gauge | Minimum G_{f} |
|---|---|
| 18 | 3.87E6 |
| 20 | 4.35E6 |
| 22 | 5.80E6 |
| 24 | 8.38E6 |

Fluid flow through a tubular fluid pathway can be analyzed using Poiseuille's equation:

$$Q = \frac{π {D}^{4} Δ P}{128 μ L} = \frac{Δ P}{{R}_{f}}$$

where ΔP is a change in pressure gradient across the length of the fluid pathway, D and L are the inner diameter and length, respectively, of the fluid pathway, μ is the viscosity of a fluid, and

$${R}_{f} = \frac{128 μ L}{π {D}^{4}}$$

is the fluid resistance. Since μ is the viscosity of the fluid and not part of the flow restriction device 60 geometry, a geometric factor G_{f} is defined such that R_{f} (the fluid resistance) is

$${R}_{f} = \frac{128 μ}{π} {G}_{f} ,$$

where

$${G}_{f} = \frac{L}{{D}^{4}} .$$

In some aspects or embodiments, the optimized fluid pathway may have multiple sections with lengths (L1, L2, L3) and inner diameters of (D1, D2, D3), the geometric factor is then:

$${G}_{f} = \frac{L 1}{D {1}^{4}} + \frac{L 2}{D {2}^{4}} + \frac{L 3}{D {3}^{4}}$$

In some aspects or embodiments, the optimized fluid pathway may have inside diameter that changes over the length of the tube, the geometric factor is then:

$${G}_{f} = {\int }_{0}^{L} \frac{dl}{{D ( l )}^{4}}$$

In some aspects or embodiments, the optimized fluid pathway may have a cross section that is not circular or complicated inside diameter profile. The geometric factor can be determined by measuring the flow rate (Q) at given pressure (ΔP) with known viscosity (μ) fluid:

$${G}_{f} = \frac{π Δ P}{128 μ Q}$$

In one aspect or embodiment, the geometric factor, G_{f}, of the flow restriction device 60 is increased to reduce a blood collection flow rate.

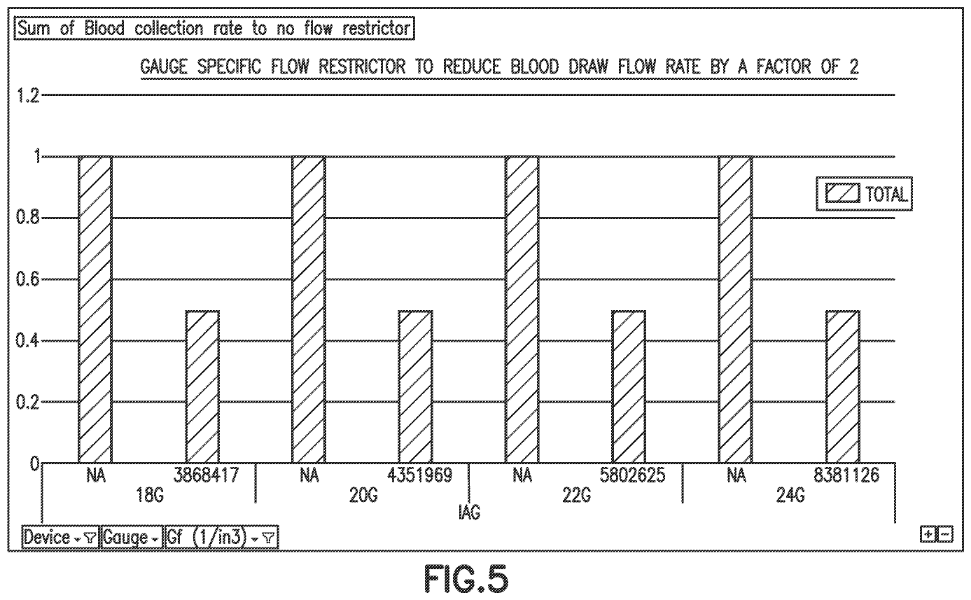

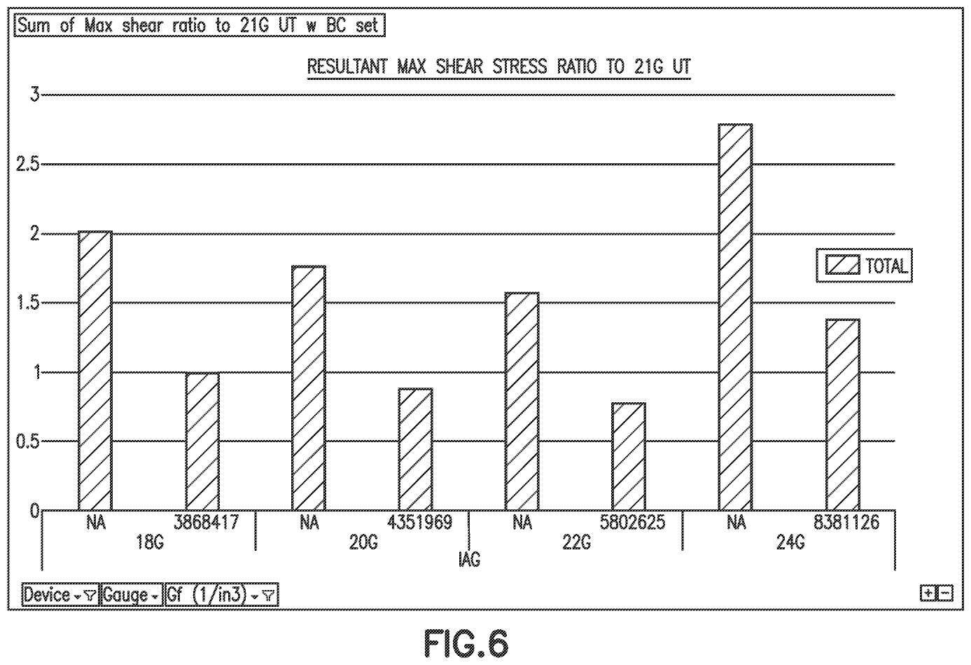

Referring to FIGS. 5 and 6, by selecting a predetermined geometric factor for the flow restriction device 60, an average blood collection flow rate can be reduced by a factor of 2. Further, the resultant max shear stress as a ratio to a max shear stress of a known 21G blood collection needle, such as the 21G Ultratouch™ blood collection set available from Becton, Dickinson and Company, is the same or less than that of the known 21G blood collection needle (except 24G) thereby indicating reduced hemolysis risk.

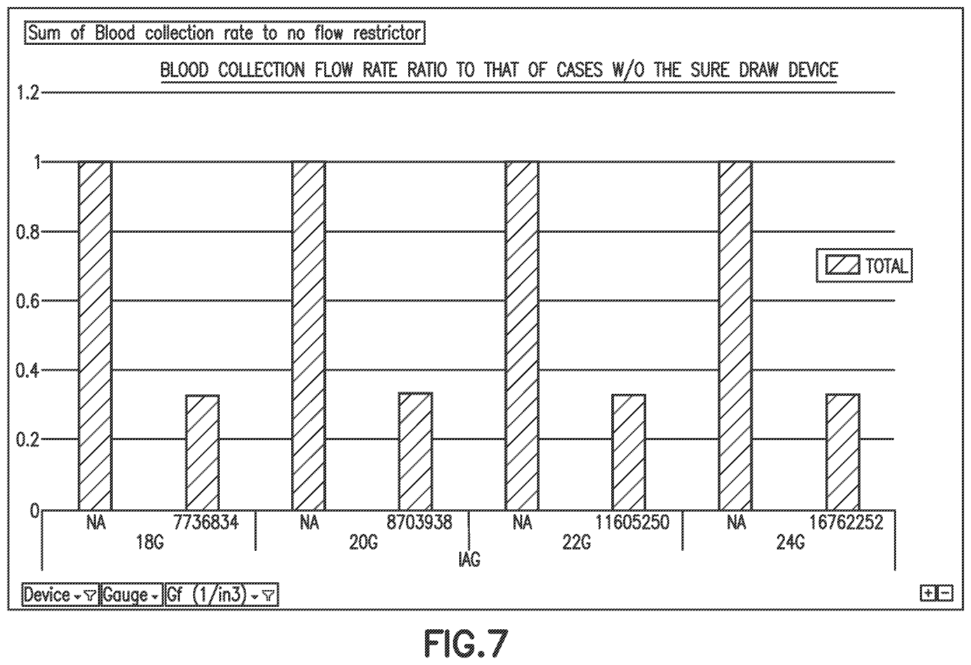

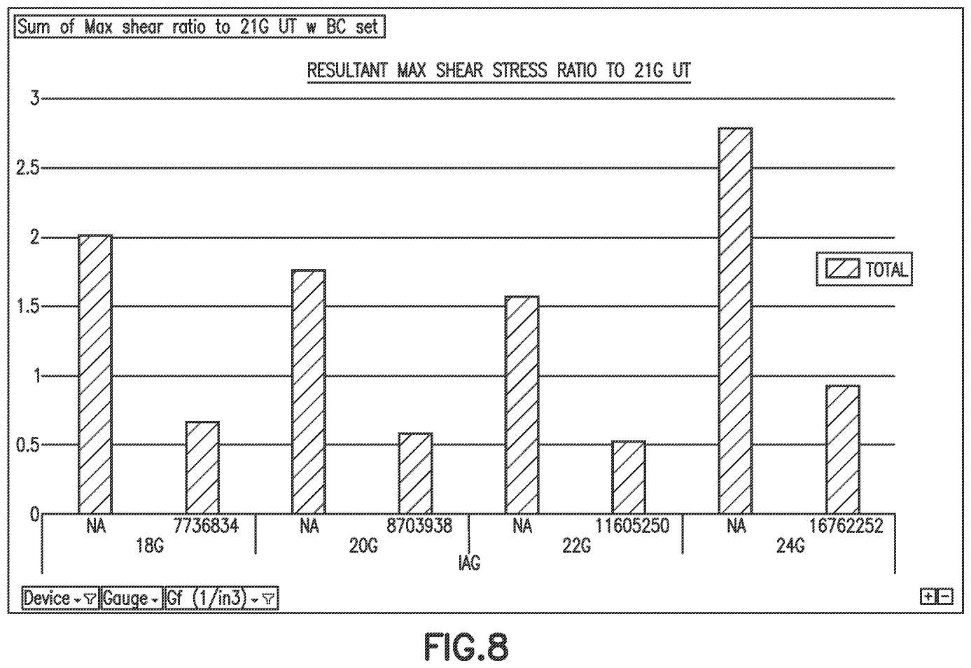

Referring to FIGS. 7 and 8, in one aspect or embodiment, gauge specific geometric factor values can be determined to achieve a 3× reduction in blood collection flow rate. The resultant max shear stress as a ratio to a max shear stress of a known 21G blood collection needle, such as the 21G Ultratouch™ blood collection set available from Becton, Dickinson and Company, is the same or less than the known 21G blood collection needle.

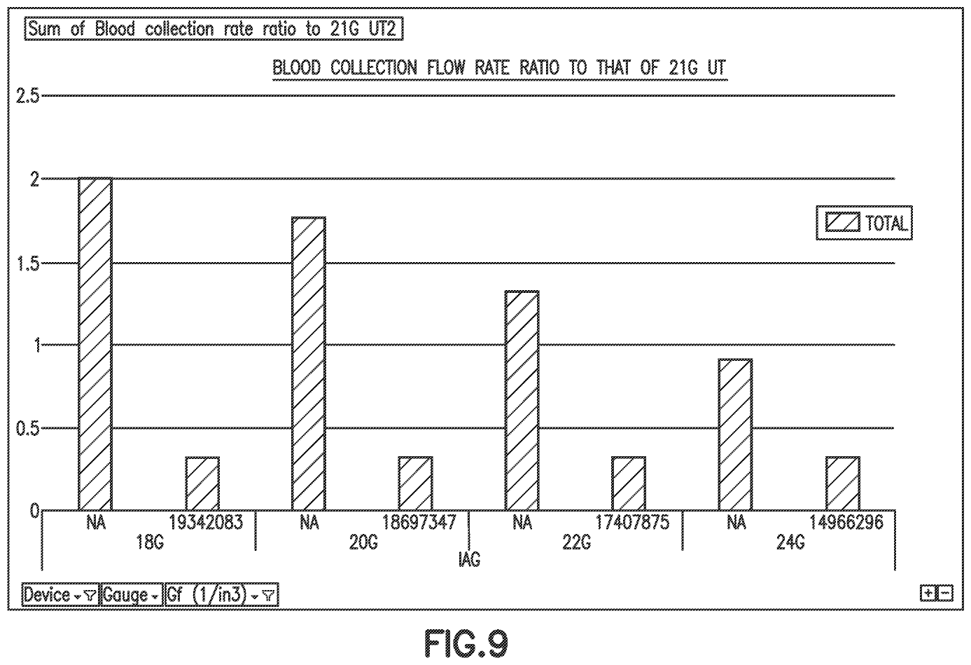

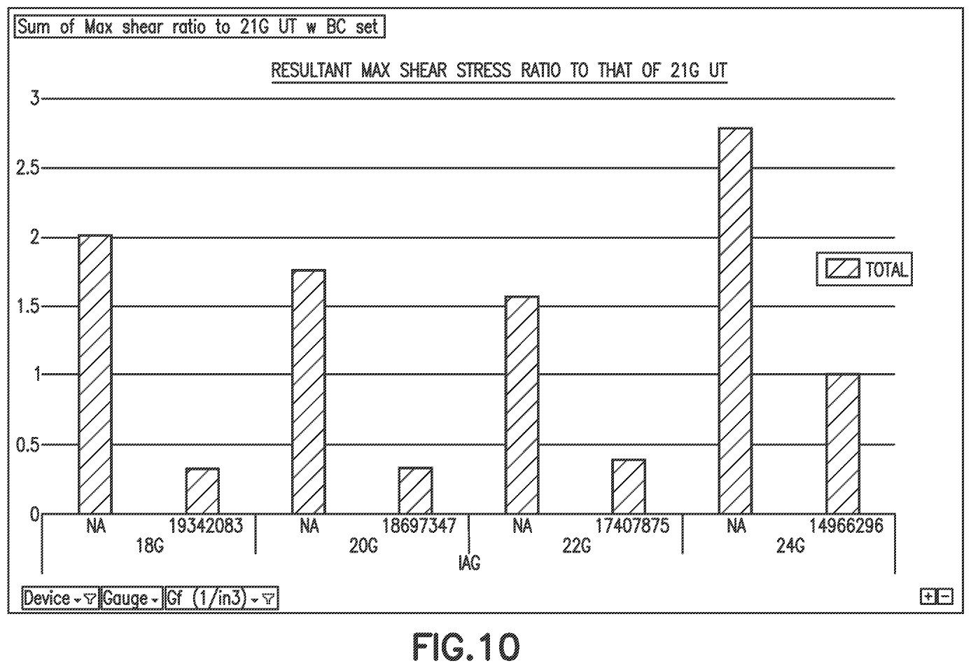

Referring to FIGS. 9 and 10, in one aspect or embodiment, gauge specific geometric factor values can be determined to achieve the same blood collection flow rate for all gauges. As shown in FIG. 9, geometric factor values for the flow restriction device can be determined to have a blood collection rate for all gauges to be 3× lower compared to a known 21G blood collection needle, such as the 21G Ultratouch™ blood collection set available from Becton, Dickinson and Company. As shown in FIG. 10, resultant max shear stress as a ratio to a max shear stress of a known 21G blood collection needle, such as the 21G Ultratouch™ blood collection set available from Becton, Dickinson and Company, is the same or less than the known 21G blood collection needle.

In one aspect or embodiment, the gauge specific geometric factor values are determined to achieve the same blood collection flow rate for all gauges with any desired lower flow rate. Similarly, the gauge specific geometric factor values can be determined to achieve any factor of reduction in blood collection flow rate for each gauge.

In one aspect or embodiment, the vascular access system 10 includes multiple blood collection devices, each including the flow restriction device 60 and each having a different flow rate. One of the multiple blood collection devices can be selected based on a patient's condition to enable improved blood draw results. In one aspect or embodiment, a method of selecting a blood collection device 50 for use with the catheter adapter 14 includes: providing a plurality of blood collection devices 50, with each blood collection device 50 including the flow restriction device 60 having a predetermined blood collection flow rate; and selecting one of the plurality of the blood collection devices 50 based on an estimated blood supply flow rate of a patient's blood vessel. In one aspect or embodiment, three blood collection devices 50 having three different blood collection flow rates may be provided, with a suitable blood collection device 50 being selected such that the blood collection flow rate of the blood collection device 50 does not exceed a blood supply flow rate of a patient's blood vessel.

Accordingly, the blood collection device 50 of the present application is configured to increase a blood collection success rate in cases where blood flow is limited. The blood collection device 50 of the present application is also configured to reduce the likelihood of vessel collapse and improve blood collection success in cases where the catheter tip was close to a valve or vein wall. Further, the blood collection device 50 of the present application is configured to reduce max shear stresses to minimize the likelihood of hemolysis during blood draw.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A vascular access system comprising:
a catheter adapter comprising a body and a catheter configured to be inserted into a patient's vasculature, the body having a distal end and a proximal end positioned opposite the distal end; and
a blood collection device in fluid communication with the catheter, the blood collection device comprising a flow restriction device configured to reduce a blood collection flow rate to be equal to or less than a blood supply flow rate of a patient's blood vessel.

2. The vascular access system of claim 1, wherein the flow restriction device comprises a flexible extension set including tubing.

3. The vascular access system of claim 2, wherein an inner diameter and length of the tubing is configured to reduce the blood collection flow rate.

4. The vascular access system of claim 1, wherein the flow restriction device is configured to reduce a max shear stress compared to a max shear stress of the catheter.

5. The vascular access system of claim 1, wherein the flow restriction device has a geometric factor, G_{f}, configured to deliver a predetermined blood collection flow rate reduction, wherein Gt=L/D⁴, wherein L is a length of a flow path of the flow restriction device, and wherein D is an inner diameter of the flow restriction device.

6. The vascular access system of claim 5, wherein the geometric factor, Gf, is at least 3.87 E6 to 8.38 E6.

7. The vascular access system of claim 5, wherein the geometric factor, Gf, is at least 3.87 E6.

8. The vascular access system of claim 5, wherein the geometric factor, Gf, is at least 4.35 E6.

9. The vascular access system of claim 5, wherein the geometric factor, Gf, is at least 5.80 E6.

10. The vascular access system of claim 5, wherein the geometric factor, Gf, is at least 8.38 E6.

11. The vascular access system of claim 1, wherein the blood collection device comprises a luer lock access device configured to receive an evacuated blood collection container.

12. The vascular access system of claim 1, wherein the blood collection device comprises a luer connector configured to be connected to a syringe barrel.

13. The vascular access system of claim 1, further comprising an instrument advancement device coupled to the catheter adapter, wherein the instrument advancement device comprises an instrument, and wherein the instrument advancement device is configured to advance the instrument from a retracted position to an advanced position beyond a distal end of the catheter.

14. The vascular access system of claim 13, further comprising an advancement member configured to be grasped by a healthcare technician, wherein movement of the advancement member moves the instrument between the retracted position and the advanced position.

15. A vascular access system comprising:
a catheter adapter comprising a body and a catheter configured to be inserted into a patient's vasculature, the body having a distal end and a proximal end positioned opposite the distal end;
a first blood collection device configured to be in fluid communication with the catheter, the first blood collection device comprising a first flow restriction device configured to reduce a blood collection flow rate to a first predetermined blood collection flow rate; and
a second blood collection device configured to be in fluid communication with the catheter, the second blood collection device comprising a second flow restriction device configured to reduce a blood collection flow rate to a second predetermined blood collection flow rate, wherein the first predetermined blood collection flow rate is larger than the second predetermined blood collection flow rate.

16. The vascular access system of claim 15, wherein the first flow restriction device comprises a flexible extension set including tubing, and wherein the second flow restriction device comprises a flexible extension set including tubing.

17. A blood collection device comprising a flow restriction device configured to reduce a blood collection flow rate to be equal to or less than a blood supply flow rate of a patient's blood vessel.

18. The blood collection device of claim 17, wherein the flow restriction device comprises a flexible extension set including tubing.

19. The blood collection device of claim 18, wherein an inner diameter and length of the tubing is configured to reduce the blood collection flow rate.

20. The blood collection device of claim 17, wherein the flow restriction device is configured to reduce a max shear stress compared to a max shear stress of the catheter.
